(19) United States
(12) Patent Application Publication
(10) Pub. No.: US 2014/0249397 A1
(43) Pub. Date: Sep. 4, 2014

(54) DIFFERENTIAL NON-CONTACT BIOPOTENTIAL SENSOR

(71) Applicant: Thalmic Labs Inc., Kitchener (CA)

(72) Inventors: Stephen Lake, Kitchener (CA); Alborz Rezazadeh Sereshkeh, Kitchener (CA); Matthew Bailey, Kitchener (CA); Aaron Grant, Kitchener (CA)

(73) Assignee: Thalmic Labs Inc., Kitchener (CA)

(21) Appl. No.: 14/194,252

(22) Filed: Feb. 28, 2014

Related U.S. Application Data
(60) Provisional application No. 61/771,500, filed on Mar. 1, 2013.

(51) Int. Cl. A61B 5/0492; A61B 5/00

(52) U.S. Cl. CPC A61B 5/0492; A61B 5/7203
USPC 600/386; 600/546

(57) ABSTRACT

A differential non-contact sensor system for measuring biopotential signals is described. The sensor is a low-noise, non-contact capacitive sensor system to measure electrical voltage signals generated by the body comprising two capacitive electrodes and outputting a differential signal.

### FIELD OF THE INVENTION

The present invention pertains to differential non-contact biopotential sensors for sensing voltage signals generated by the body with a single sensor, and methods of use thereof.

### BACKGROUND

Electromyography, or EMG, is a technique for measuring muscle response to nervous stimulation. EMG signals are generally measured using either surface electromyography (sEMG) or intramuscular EMG. Intramuscular EMG techniques are invasive, requiring insertion of a needle or electrode through the skin. Surface EMG is commonly collected using sensors placed on the surface of the skin, nearby the underlying muscle. Current sensors for measuring sEMG require direct electrical contact with the skin. The most outward layer of skin, the stratum corneum, is generally dry and offering low electrical conductivity. Therefore, to obtain a low-impedance electrical connection with the skin abrasion and/or conductive gels are applied to the skin below the electrodes. Applying a gel is both inconvenient to the subject and not practical for many situations where one may desire to measure biopotential signals. For each application of electrodes to the subject the site is shaved to remove hair and abrade the skin and an electrode with gel is placed on the skin. When removed, the conductive gels leave behind a residue which requires further cleaning In applications where these signals are measure frequently, for example in a muscle-computer interface or for the monitoring of athletic performance, this process is impractical and problematic.

There have been many attempts to create sensors which do not require skin preparation or conductive gel but instead rely on dry contact with the skin, such as in U.S. Pat. Nos. 5,003,978 and 6,510,333, both incorporated herein by reference. These types of dry contact sensors suffer from fluctuating signal magnitudes as the subject perspires or as the pressure on the sensor varies. Often when first applied to dry skin, the output signals from dry contact electrodes are low in magnitude and noisy and then change after the electrode is left in contact with the skin for some time.

More recently attempts at non-contact biopotential sensing have been made such as in U.S. Pat. No. 8,054,061, incorporated herein by reference. Non-contact approaches offer the advantage of being able to sense biopotential signals without skin preparation and with consistent signals less dependant on the current skin conditions such as perspiration or abrasion. Therefore, a non-contact sensor is most desirable.

The subject's body and surrounding environment generally are very noisy due to emissions from power lines, computers, electrical equipment, radio frequency devices, and others. Often the noise measured on the skin of the subjects body is an order of magnitude or more higher than the biopotential signals which one desires to measure. For this reason multiple sensors are used, and in a later stage of processing the signals are subtracted to attempt to eliminate noise that is common to two or more sensors. In measurement of EMG signals, for example, two distinct sensors are commonly placed 20 - 40mm apart, along the direction of the underlying muscle, each sensor independently measuring the signal at its location. The outputs of these two sensors are later subtracted, either by analog circuitry or digital computations, to reduce noise and reduce the output to the biopotential signal of interest. It is desirable to increase the signal to noise ratio (SNR) to yield the best possible output signal of the system.

In a biopotential sensor system, using two distinct sensors is not desirable because each sensor, as well as any filtering and amplification circuitry, and the interconnections between the sensors, adds noise to the system which may vary from electrode to electrode. Therefore when the signals are finally subtracted to produce a differential measurement between the two electrodes much noise remains. Attempts to reduce solve this problem by creating a differential sensor with two or more electrodes have been successful for gel-based and dry-contact electrodes, such as in the differential electrode from Delsys™. However, these electrodes still suffer from the same limitations as other dry-contact sensors mentioned above.

There remains a need for a differential non-contact biopotential sensor which provides a high signal to noise ratio and outputs an amplified version of the underlying biopotential signal.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a differential non-contact sensor system for measuring biopotential signals. The sensor comprises two capacitive electrodes and outputs a differential signal, which allows for better performance. The presently described sensor is a low-noise, non-contact capacitive sensor system to measure electrical voltage signals generated by the body.

In one aspect there is provided a sensor for sensing electrical voltage signals from a muscle or a portion of a muscle in a body of a subject, the sensor comprising: at least two conductive elements for capacitively coupling to the body of the subject; and an amplifier; wherein the amplifier: receives measurement signals from the at least two conductive elements; rejects signals common to the at least two conductive elements to obtain a differential signal; and amplifies the differential signal.

In one embodiment, the at least two conductive elements are configured to be positioned adjacent to but not in contact with the body of the subject. In one preferred embodiment, the at least two conductive elements are two sensing plates. Preferably the at least two conductive elements comprise two conductive elements. Other preferred configurations comprise 4, 6 or 8 conductive elements, with multiple elements connected to each amplifier input.

In another embodiment, the sensor further comprises a high-pass filter to filter the differential signal.

In another embodiment, the sensor further comprises a shielding element surrounding at least one of the at least two conductive elements. In one preferred embodiment, the shielding element is a shield trace or a shielding ring.

In another embodiment, the sensor further comprises an analog circuit downstream the amplifier. In one preferred embodiment, the analog circuit comprises a gain block, a low-pass filter, an anti-aliasing filter, or a combination thereof.

In another embodiment, the sensor further comprises a casing.

In another embodiment, the sensor further comprises the differential signal is transmitted to a detector. In one preferred embodiment, the detector is an analog detector or a digital detector. In another preferred embodiment, the detector records, analyzes, or visualizes the differential signal.

In another aspect, there is provided a method of sensing electrical voltage signals from a body of a subject, the method comprising: capacitively coupling two conductive elements to a body, wherein changes in electric potential on or in the body generate an electric field that induces change in the electric potential of the conductive elements; generating an input signal for each of the two sensing elements, wherein the input signals are based on the electrical voltage signal detected by each sensing plate; and receiving measurement signals from the sensing elements as an input and outputs an amplified signal corresponding to the difference in the input signals.

In one embodiment of the present method, the two conductive elements are not in direct contact with the body of the subject. In one preferred embodiment, the sensor is placed less than about 10 mm away from the subject.

In another embodiment, the method optionally comprises placing an external shield adjacent the body of the subject to reduce environmental or ambient noise.

### BRIEF DESCRIPTION OF THE FIGURES

For a better understanding of the present invention, as well as other aspects and further features thereof, reference is made to the following description which is to be used in conjunction with the accompanying drawings, where:

FIG. 1 depicts an example of a differential non-contact sensor system;

FIG. 2 depicts an example circuit implementing the system of FIG. 1;

FIG. 3A is a perspective view of an example of a differential non-contact sensor implemented on a printed-circuit board, and FIG. 3B is a side cross-sectional view of the same sensor;

FIG. 4 graphically depicts example output data measuring muscle activity using of the system of FIG. 1;

FIGS. 5A-5C show three examples of sensing plate and shield configurations;

FIG. 6 illustrates a method of measuring an electric field using a capacitive sensor system according to an embodiment; and

FIG. 7 is a sketch of the positioning of the optional external shield relative to the sensor adjacent the body of a subject.

### DETAILED DESCRIPTION OF THE INVENTION

Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

The term “non-contact” as used herein means that the biopotential sensor is not required to be in contact with the body of the subject, however it is understood that the sensor may be also be used as a contact sensor if desired.

Biopotential Sensor

The present application describes a differential non-contact biopotential sensor for measuring biopotential signals and a method of use thereof. The non-contact capacitive sensor does not require skin surface contact with the subject, operates by capacitive coupling, and is capable of measuring electric fields through hair, clothing or other skin coverings. The sensor has two sensing plates from which a differential signal is measured using capacitive coupling to the body of the subject. The sensor offers enhanced performance from other types of capacitive physiological sensors through the use of differential measurement directly at the signal source, which allows for a reduction of noise sensed from the subject's body.

Capacitive coupling, in this context, allows an induction of charge at a capacitive element of the sensor that corresponds to electrical signals from or through the skin of a subject. The insulating layer between the electrodes and the skin (or the air gap) forms an insulator, between the charges skin and plates, thereby forming a parallel-plate capacitor. The electrical voltage at the surface of the skin is thereby sensed from this coupling. Since there is a difference in voltage between the surface of the skin and the electrode plates, an electric field is formed between them.

The present differential capacitive biosensor system requires that only a single sensor be affixed over the area of interest for sensing physiological signals, such as the muscle in EMG applications. The sensor includes two conductive plates which couple capacitively with the skin of the subject to measure electrical signals within and below the skin, requiring no direct contact with the subject's body. The electric potential of the plates then corresponds to the electrical potential present across the site of interest within the subject. An instrumentation amplifier receives the measurement signals from the conductive elements as an input and outputs an amplified signal corresponding to the difference in the input signals. The amplifier rejects signals common to both plates, i.e. the noise, and amplifies the differential signal. A bias current return path is provided to prevent the plates from charging up due to bias currents. The output of the amplifier system may be optionally further amplified and/or filtered and connected to an analog or digital system to record, analyze, or visualize the biopotential signal.

The sensor requires no direct electrical connection to the subject's body. For example, an insulator may be present, or may be placed between the conductive sensing plates and the subject's body. In some embodiments this insulator may include an air gap of 1-20 mm or more. In others a thin layer of insulating material is present between the sensing plates and the body, for example a layer of solder mask applied over the sensing plates.

The output of the system may be connected to another system in order to use, process or display the physiological signal. In some embodiments the analog output signal will be digitized through the use of an analog-to-digital converter (ADC), either directly on the sensor or at some downstream location connected to the sensor output. The output signal may be used, for example, by a Kinesiologist to observe muscle activation patterns, or as another example, in a muscle-computer interface to control software, robots, or other digital equipment from sensed muscle activity. The sensor may also be used to measure other physiological signals such as heart rate or neural activity, each of which may be used in a multitude of applications.

One exemplary use of the present capacitive biosensor is in the detection and diagnosis of muscular and neuromuscular diseases in which the function of muscle is affected by the disease. Some muscular diseases which may be detected by a change in electrical voltage output of muscle include cerebrovascular accident (stroke), Parkinson's disease, Multiple sclerosis, Huntington's disease (Huntington's chorea), amyotrophic lateral sclerosis and Creutzfeldt-Jakob disease. Muscular atrophy can occur over a period of time, and can be detected by punctuated screening. Athletes, bodybuilders, as well as people with a general interest in healthy muscle growth, optimization and function can find uses of the present sensor in the detection of muscle growth and change as a result of physical activity.

It is envisaged that the present sensor can be used independently, but may also be incorporated into smart clothing, sports equipment, jewellery, watches, eyeglasses, hats and shoes. The present biosensor may also be useful in the control of prosthetic devices. In one example, local muscle movement at the site of an amputation detected by the sensor can be calibrated to enable control of a prosthetic device. Another use of the present sensors is in the remote operation of electronic devices such as computers, gaming systems, or any device that may be controlled by way of gestural input.

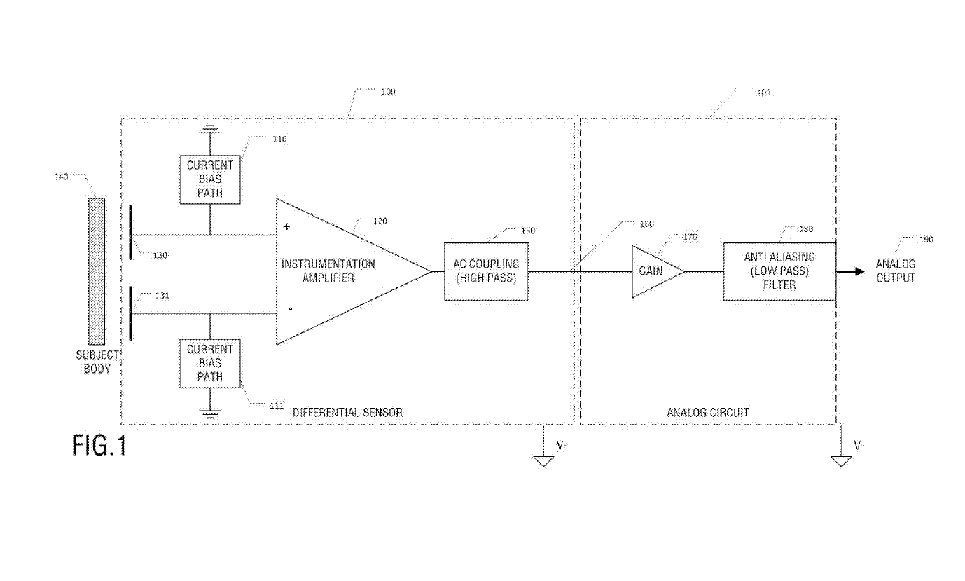

A block diagram of one embodiment of the present biosensor system is shown in FIG. 1. In this embodiment the sensor 100 is electrically connected to an (optional) analog processing circuit 101 to further amplify and filter the signal. The differential sensor comprises first and second sensing plates 130 and 131 spaced apart from one another and shown adjacent the body of a subject 140. A path for bias current from the amplifier to return to ground is shown as current bias paths 110 and 111, wherein the first current bias path 110 is connected between the first sensing plate 130 and the amplifier 120, and the second current bias path 111 is connected between the second sensing plate 131 and the amplifier 120. The current bias path elements 110 and 111 prevent charging up the sensing plates. Amplifier 120 is a differential amplifier which amplifies the differences in potential at each input + and − from the first and second sensing plates 130 and 131, respectively.

Generally the amplifier 120 will be an amplifier configured to amplify the electrode signals differentially from the conductive elements. One preferred amplifier is an instrumentation amplifier, since these amplifier configurations have high input impedance. Another preferred amplifier is an operational amplifiers, preferably one packaged in a single integrated circuit. The amplifier can also have other arrangements, such as differential amplifiers. In the case of more than two inputs or conductive elements, one can create a “double differential” electrode with three sensor plates, which would require a configuration with two instrumentation amplifiers and a third differential amplifier to combine the signals. Although the figures show two conductive elements, other configurations can include more conductive elements. Alternate preferred configurations can include 4, 6 or 8 conductive elements. An optional high pass filter 150 is adapted to remove DC offset, thereby AC-coupling the signal. For example, a preferred filter would operate in the range of 0.1-10 Hz, and preferably around 2 Hz. The purpose of the high pass filter is to remove any DC offset (i.e. “AC Couple” the signal) from the amplifier while minimizing distortions to the EMG waveform, which contains information in the range 0-500 Hz, with most concentrated in the range of 50-150 Hz.

The differential sensor output 160 can optionally be connected to an analog processing circuit, or analog circuit 101. The analog circuit 101 comprises an optional additional gain block 170, an optional low-pass filter or anti-aliasing filter 180, or both. The low-pass/anti-aliasing filter is selected depending on the sampling rate and characteristics of the analog to digital converter (ADC), if used. Since the EMG information is generally in the range 0-500 Hz, this low-pass filter can set as high as 500 Hz to minimize high frequency noise aliasing into the Nyquist range of the sampled data. The gain block is preferably a gain in the range of 10-10,000×, which is determined depending on the application and configuration. The output of the system can be further connected to an analog to digital converter to digitize the signal for processing, but could also be connected to an analog display or other device. Analog circuit 101 may or may not be placed directly nearby the sensor electronics. In general amplifier 120 should be placed as close to the sensing plates as possible, however the additional components may be located on a secondary circuit board or assembly. The output of the differential sensor can also be read directly at the output of amplifier 120, or at the output of high-pass filter 150 without the further amplification and filtering. However, in the preferred embodiment, the analog circuit 101 comprising the gain block 170 and anti-aliasing filter 180 are present to improve the quality of the output signal. The output of the sensor may be connected to an analog or digital system to record, analyze, or visualize the biopotential signal, such as in a muscle-computer interface.

The sensing plates 130 and 131 can be made of any conductive material. Non-limiting examples of exemplary conductive material that may be used for the sensing plates are copper, aluminum and silver, with copper being a preferred conductive material, for example. Both the two dimensional and three-dimensional shape will contribute to performance. A significant determinant to performance is the two-dimensional area of the sensing plates. The sensors behave as parallel plate capacitors, and therefore it follows that surface area (A) directly affects sensitivity. The sensing plates could be very small, for example 5 mm diameter circles, or very large, for example 50 mm×50 mm squares. In the present system, a surface area of approximately 200 mm² has been found to be preferable to provide good performance. The shape of the sensing plates will also determine primarily the area of the skin contributing to the sensed signal. For example, a larger electrode will be more sensitive but provide less location-specific signal. In other words, for larger sensing plates, activity from more muscles may be included in the obtained signal. The selection of the surface area of the sensing plates will vary based on the desired application of the sensor.

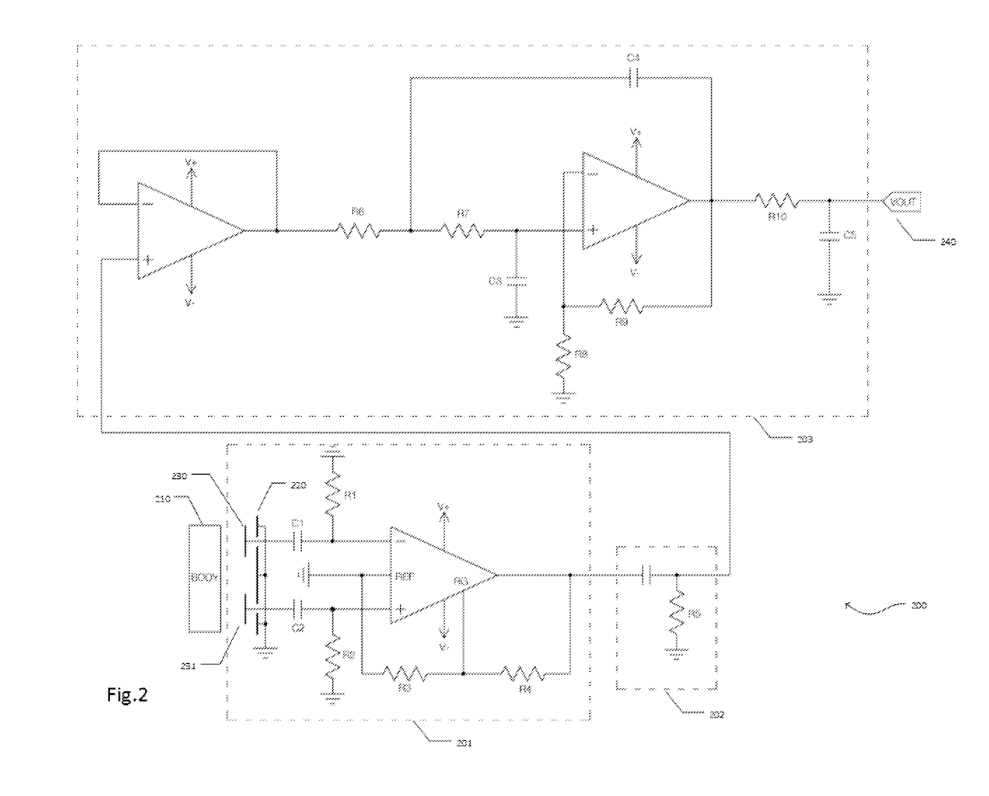

FIG. 2 depicts a schematic diagram of one exemplary implementation of the sensor system shown in FIG. 1. In FIG. 2, the sensor 200 comprises a differential sensor 201. Also shown in FIG. 2 is an optional high-pass (AC-coupling) filter 202, which can be located on the sensor itself, or can also be located on a separate physical circuit board, along with the other optional components. Analog circuit 203 is coupled to the output of high-pass filter 202 to amplify and filter the output signal from the differential sensor 201 and high-pass filter. First and second sensing plates 230 and 231 are placed in proximity to the body of a subject 210. An optionally shield plane 220 is shown located above the sensing plates 230 and 231. The shielding plates help to reduce the effects of noise from the environment or other electronics located near the electrode. For example, 60 Hz noise from powerlines, lights, etc. is reduced with the use of shielding material. The shield plate can be, for example, a solid layer of copper, or a hatched layer, also known as a hatched ground plane. Preferably a hatched pattern is used to reduce capacitive coupling to the sensing plates, which reduces the relative coupling to the subject and decreases performance. The thickness of the shielding plate or shielding material can vary. Normally a copper layer within the PCB stack is selected as a ground and filled with a hatch pattern. A common copper thickness for PCB manufacturing is 1 oz. copper, however other thicknesses can be used.

Circuit output 240 transfers the processed signal to a computing or processing device. The sensor circuit output 240 corresponds to the physiological signals from within the subject's body, and may also include noise from the environment or the electronics. Additional components to measure noise from the environment and optionally cancel the detected noise from the output signal may also be incorporated into the sensor.

Alternative electronic configurations are not excluded from the present sensor. In one alternative, the instrumentation amplifiers could be replaced by other components, such as standard operational amplifiers arranged in a differential configuration. Additional filters could also be added before or after the amplifier. The analog circuit (101/203) may be omitted entirely or may be replaced with many other configurations of filters and/or additional gain to post-process the signal from the sensor. The high pass filter (150/202) is also optional. The bias return path (shown as 110 and 111 in FIGS. 1, and as R1 and R2 in FIG. 2) may not need to be explicitly included if the leakage current in the PCB construction is sufficient to prevent the charging of the sensor plates. C1 and C2 in FIG. 2 are also optional and not required for operation. Various ground reference schemes known to those skilled in the art may be employed, such as a right-leg-drive type circuit.

An optional casing or casing shield can be used to partially or completely encapsulate the sensor to protect the sensor from the damage of normal use. The casing can have the effect of reducing noise from other devices and the environment to the sensor. This type of casing shield can be an external enclosure which isn't part of the PCB. The casing can be coated with or made with a metallic or conduction plate placed above the electrode, or a case enclosing the electrode which help to shield it from interference/noise. The casing can be made from any material compatible with the sensor, including polymers such as plastic, films, foams, and other materials. A metallic material may also be used in the casing, which could be used to aid in shielding the electrodes.

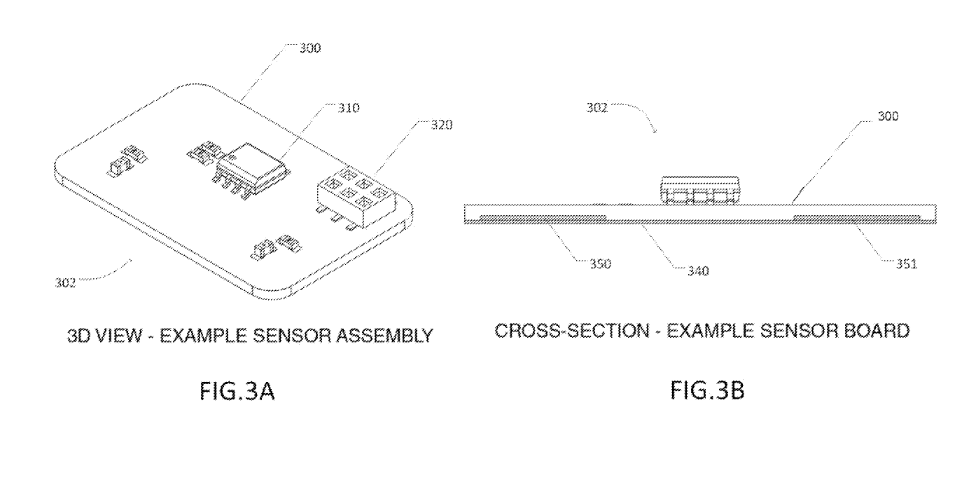

FIGS. 3A and 3B depicts a mechanical drawing of one embodiment of the system shown in FIGS. 1 and 2. FIG. 3A is a perspective view of one exemplary embodiment of the sensor 302, and FIG. 3B is a side cross-sectional view of the same sensor device. In one preferred embodiment, the sensing plates are traces on the bottom of a printed circuit board (PCB) 300. As shown, the PCB 300 houses the electronics and sensing plates 350 and 351 of the sensor 302. The PCB 300 can be made from Fiberglass, such as, for example FR4, a standard PCB material. In the embodiment shown, the sensing plates 350 and 351 are coated in a non-conductive solder mask material 340. The solder mask acts as an insulator to insulate the sensing plates from the skin of the subject, thereby allowing a charge to build on the plates and them to act as capacitors. Any other insulating material could be used, such as, for example, plastic, foam, other polymers or fabric. A material with a high dielectric constant is preferred, but not required. Passive components 330 are also shown in FIG. 2 as C1, R1. Instrumentation amplifier 310 is shown on the second face of the PCB in FIG. 3A. An output interface connector 320 shown in FIG. 3A can be connected to a peripheral device for processing or analysing the output signal from the sensor 302. As shown in FIG. 3B, sensing plates 350 and 351 are located on the first face of the PCB which will come into proximity of the body of a subject, and the other bulkier electronic components are located on the opposite or second face of the PCB.

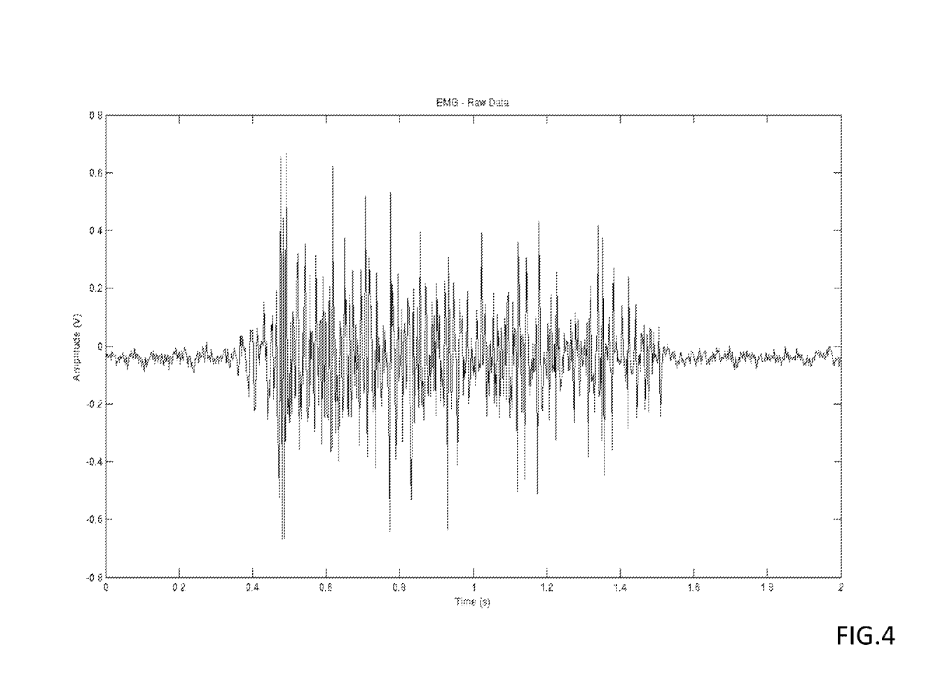

FIG. 4 graphically depicts an example of data recorded from the output of the sensor system 200 shown FIG. 2. Raw data is shown as measured by an analog to digital converter connected to the output of the system. In this example the sensor is placed near the surface of a subject's skin above a muscle, and the muscle is contracted, producing the output shown.

Further improvements may be realized by adding shielding around the plates to reduce pickup of noise from the environment. In another embodiment a conductive ring is present around the electrodes, similarly held at a constant reference voltage. In some other embodiments, both a shielding ring and a conductive surface are used to shield the two plates from noise.

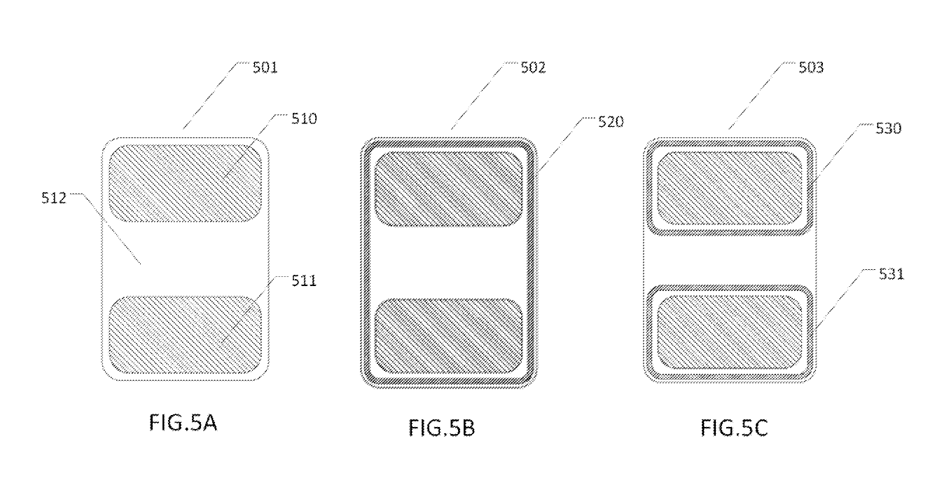

FIGS. 5A-5C show three examples of sensing plate and optional shield or shielding ring configurations. In FIG. 5A, sensor 501 is shown with first sensing plate 510 and second sensing plate 511 as two rectangular pads 10 mm×20 mm, 20 mm, center to center spacing. No additional shield traces are shown. A nonconductive base material 512 is shown, and can be, for example, a FR4 Fiberglass PCB. FIG. 5B shows a sensor 502 with a configuration similar to that shown in FIG. 5A, but with a single shield trace 520 added to reduce noise. FIG. 5C shows a sensor 503 configuration similar to that shown in FIG. 5B, but with two shield traces 530 and 531, one surrounding each sensing plate. The shield traces shown in FIGS. 5B and 5C are connected to a ground or reference voltage. The sensing plates may also be various sizes and shapes.

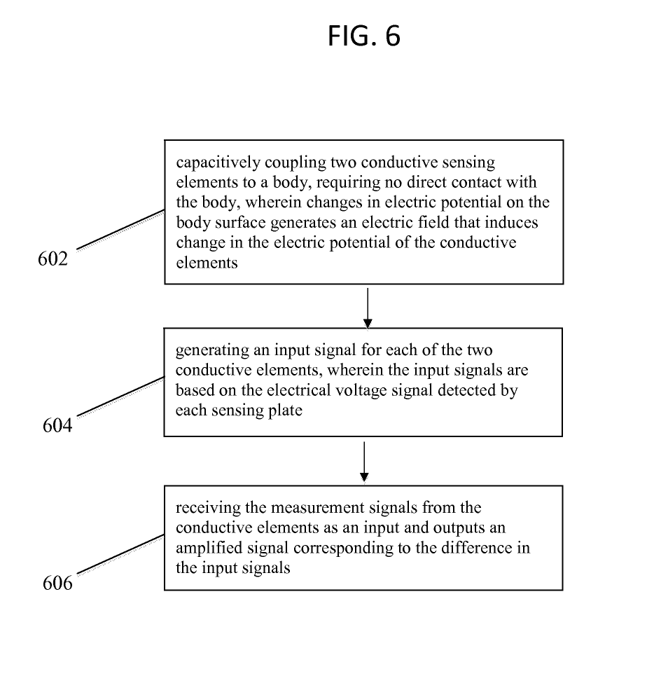

FIG. 6 is a schematic representation of method steps carried out by the present sensor. In the first step 602, the sensor capacitively couples the two conductive elements to the body of a subject, requiring no direct contact with the body, wherein changes in electric potential on the body surface generates an electric field that induces change in the electric potential of the conductive elements. In the second step 604, the sensor generates an input signal for each of the two conductive elements, wherein the input signals are based on the electrical voltage signal detected by each of the two sensing plates. In the third step 606, the sensor receiving the measurement signals from the sensing elements as an input and outputs an amplified signal corresponding to the difference in the input signals.

The electrodes can operate directly against the body, and can be placed close to or against the skin (<1 mm). The electrode may also be placed away from the body and do not require direct contact with skin. Preferably, the sensors are placed no more than 10 mm away from the skin surface. In general, the sensor does not require an electrical connection to skin as required by traditional electrodes.

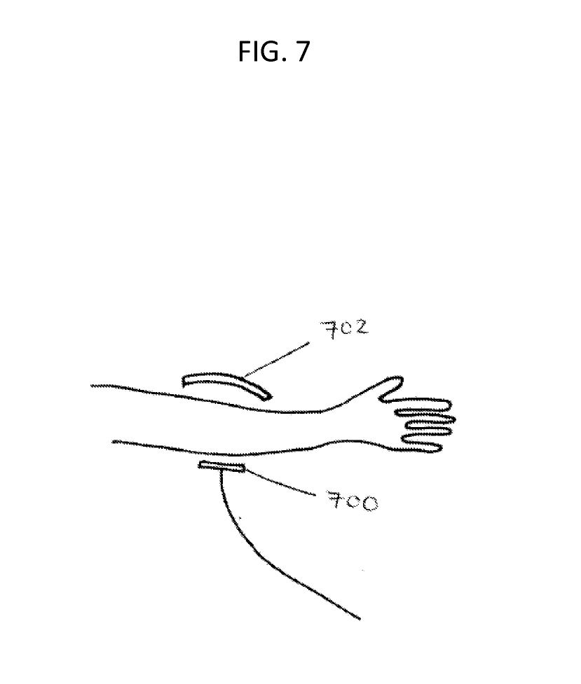

As shown in FIG. 7, an optional external shield 702 could also be placed on the opposite side of the sensor 700, away from the subject. This optional external shield can also serve to reduce the amount of environmental or ambient noise detected by the sensor.

The present method can be practiced on a patient a single time, and single measurements or signal can be compared to baseline or reference measurements to determine muscle performance or properties at any given moment in time. Alternatively, the results of multiple sensor measurements can be collected over a period of time, and the measurements analysed to establish a trend in muscle change in a subject over time. Further, the trend observed in a subject can be compared to baseline or reference sample trends to determine muscle change in the subject. Reference single measurements or trend measurements can be collected for use as a reference for a variety of muscular diseases, and may be used to compare with the single measurements or trends of a subject to assist in the diagnosis of these diseases.

All publications, patents and patent applications mentioned in this Specification are indicative of the level of skill of those skilled in the art to which this invention pertains and are herein incorporated by reference to the same extent as if each individual publication, patent, or patent application was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims. The scope of the claims should not be limited to the preferred embodiments described.

1. An electromyography sensor comprising:
a first sensor plate;
a second sensor plate, wherein the first sensor plate and the second sensor plate are each formed of conductive material;
an amplifier that is communicatively coupled to both the first sensor plate and the second sensor plate, wherein in use the amplifier:
receives signals from the first sensor plate and the second sensor plate;
rejects signals common to the first sensor plate and the second sensor plate to obtain a differential signal; and
amplifies the differential signal;
a first current bias return path coupled between the first sensor plate and the amplifier and which couples the first sensor plate and the amplifier to a ground; and
a second current bias return path coupled between the second sensor plate and the amplifier and which couples the second sensor plate and the amplifier to a ground.

2. The electromyography sensor of claim 1, wherein in use the first and the second sensor plates are positioned adjacent to but not in contact with a body of a subject and capacitively coupled to the body of the subject.

3. (canceled)

4. The electromyography sensor of claim 1, further comprising a high-pass filter communicatively coupled to an output of the amplifier to, in use, filter the differential signal.

5. The electromyography sensor of claim 1, further comprising at least one shield at least partially surrounding at least one of the first and the second sensor plates.

6. The electromyography sensor of claim 5 wherein the shield is selected from the group consisting of: a shield trace, a shield plane, a shielding ring, and a casing.

7. The electromyography sensor of claim 1, further comprising an analog circuit communicatively coupled downstream from the amplifier.

8. The electromyography sensor of claim 7 wherein the analog circuit comprises at least one circuit selected from the group consisting of: a gain block, a low-pass filter, and an anti-aliasing filter.

9. (canceled)

10. The electromyography sensor of claim 1, further comprising a detector communicatively coupled to the amplifier, and wherein in use the differential signal is transmitted to the detector.

11. The electromyography sensor of claim 10 wherein the detector is an analog detector or a digital detector.

12. The electromyography sensor of claim 10, wherein in use the detector records, analyzes, and/or visualizes the differential signal.

13. A method of sensing electrical voltage signals from a body of a subject, the method comprising:
coupling two sensor plates to the body, wherein changes in electric potential on or in the body generate an electric field that induces changes in a respective electric potential of each of the two sensor plates;
generating a respective signal by each of the two sensor plates, wherein the respective signals are based on electrical voltage signals detected by the two sensor plates from the body;
receiving signals from the two sensor plates as inputs to an amplifier;
outputting an amplified signal from the amplifier, the amplified signal corresponding to a difference in the input signals; and
discharging the two sensor plates by respective current bias return paths that respectively connect each of the two sensor plates to ground.

14. The method of claim 13 wherein the two sensor plates are not in direct contact with the body of the subject, and wherein coupling two sensor plates to the body includes capacitively coupling the two sensor plates to the body.

15. The method of claim 14 wherein the two sensor plates are positioned less than about 10 mm away from the subject.

16. The method of claim 13, further comprising placing an external shield adjacent the body of the subject to reduce environmental and/or ambient noise.

17. The electromyography sensor of claim 1, further comprising:
a first resistor, wherein the first current bias return path includes the first resistor; and
a second resistor, wherein the second current bias return path includes the second resistor.
